(19) United States
(12) Patent Application Publication
(10) Pub. No.: US 2020/0069219 A1
(43) Pub. Date: Mar. 5, 2020

(54) Combining Pulmonary Function Test Data with Other Clinical Data

(71) Applicant: University of South Carolina, Columbia, SC (US)

(72) Inventors: Richard A. Hoppmann, Columbia, SC (US); Keith Barron, Columbia, SC (US); Robert Haddad, Columbia, SC (US); Duncan Norton, Columbia, SC (US)

(73) Assignee: University of South Carolina, Columbia, SC (US)

(21) Appl. No.: 16/559,798

(22) Filed: Sep. 4, 2019

Related U.S. Application Data
(60) Provisional application No. 62/726,549, filed on Sep. 4, 2018.

(51) Int. Cl. A61B 5/08; G16H 50/20; G16H 10/60; G16H 40/63; A61B 5/00; A61B 5/0205

(52) U.S. Cl. CPC A61B 5/0826; G16H 50/20; G16H 10/60; A61B 5/01; A61B 5/7246; A61B 5/7425; A61B 5/0205; G16H 40/63

(57) ABSTRACT

A portable device for receiving pulmonary function test data and displaying and analyzing same with both real-time and recorded patient clinical data such as that obtained with ultrasound and/or an electronic stethoscope.

### BACKGROUND OF THE INVENTION

### 1) Field of the Invention

The present invention relates to a portable device for receiving pulmonary function test data and displaying and analyzing same with both real-time and recorded patient clinical data such as that obtained with ultrasound or an electronic stethoscope.

### 2) Description of Related Art

Lung diseases are some of the most common medical conditions in the world. Tens of millions of people suffer from lung disease in the U.S. Smoking, infections, and genetics are responsible for most lung diseases. The lungs are part of a complex apparatus, expanding and relaxing thousands of times each day to bring in oxygen and expel carbon dioxide. Lung disease can result from problems in any part of this system.

Diseases that affect the airways include: asthma—airways are persistently inflamed, and may occasionally spasm, causing wheezing and shortness of breath, allergies, infections, or pollution can trigger asthma's symptoms; chronic obstructive pulmonary disease (COPD)—lung conditions defined by an inability to exhale normally, which causes difficulty breathing; chronic bronchitis, a form of COPD characterized by a chronic productive cough; emphysema—lung damage allows air to be trapped in the lungs in this form of COPD with difficulty blowing air out being a hallmark of the disease; acute bronchitis—a sudden infection of the airways, usually by a virus; cystic fibrosis, a genetic condition causing poor clearance of mucus from the bronchi wherein the accumulated mucus results in repeated lung infections.

Lung disease includes a variety of disease categories such as restrictive, obstructive, and vascular lung disease. Obstructive lung diseases are the most common and include asthma, emphysema, chronic bronchitis, and others. Emphysema and chronic bronchitis are chronic lung diseases that are commonly referred to as Chronic Obstructive Pulmonary Disease or COPD. Dyspnea is the sensation of shortness of breath and is one of the cardinal symptoms of lung disease, sometimes described as “air hunger.” There are many causes of dyspnea and these can involve the lung itself, the heart, blood, and other organ systems. While an episode of dyspnea is not always directly related to an individual's health, e.g., a person can feel short of breath after intense exercise, when traveling to a high altitude, or going through major temperature changes. However, dyspnea usually relates to health problems.

The most common causes of dyspnea are asthma, heart failure, chronic obstructive pulmonary disease (COPD), interstitial lung disease, pneumonia, and psychogenic problems that are usually linked to anxiety. If shortness of breath starts suddenly, it is called an acute case of dyspnea. Acute dyspnea could be due to: asthma; anxiety, pneumonia, choking on or inhaling something that blocks breathing passageways, allergic reactions, anemia, serious loss of blood, exposure to dangerous levels of carbon monoxide, heart failure, hypotension (low blood pressure), pulmonary embolism (a blood clot in an artery in the lung), collapsed lung(s), or hiatal hernia. If a person experiences shortness of breath for over a month, the condition is called chronic dyspnea. Chronic dyspnea could be due to: asthma; COPD; heart problems; obesity; and interstitial lung disease, a disease that causes scarring of the lung tissue.

Determining the cause of dyspnea can be quite challenging with so many potential causes and combination of causes. It is critical that the cause of the dyspnea be correctly diagnosed as early in the process as possible since the treatment can be very different for the various causes of dyspnea. For example, dyspnea could be caused by heart failure or obstructive lung disease. Dyspnea from either of these causes can be life threatening if the correct diagnosis is not made and the appropriate treatment initiated early in the clinical scenario.

Ultrasound of the heart, the lungs, and the blood vessels is becoming a common approach to assist diagnoses of many disease states, including those that cause dyspnea. Although ultrasound is excellent in identifying some causes of dyspnea such as congestive heart failure, collapsed lung, and pneumonia, it offers little help in diagnosing most of the causes due to obstructive lung disease. In addition, patients can have a history of more than one disease that can cause dyspnea and determining the underlying cause of a particular episode of dyspnea in these patients can be quite challenging.

Although listening to the lungs with a stethoscope for evidence of obstructive lung disease such as expiratory wheezes can be helpful, the definitive way to diagnose obstructive lung diseases is by assessing the patient's lung function with pulmonary function tests (PFT) or forced spirometry. Pulmonary function tests (PFTs) are noninvasive tests that show how well the lungs are working. The tests measure lung volume, capacity, rates of flow, and gas exchange. This information can help a healthcare provider diagnose and decide the treatment of certain lung disorders.

There are 2 types of disorders that cause problems with air moving in and out of the lungs. Obstructive—this is when air has trouble flowing out of the lungs due to airway resistance. This causes a decreased flow of air. Restrictive—this is when the lung tissue and/or chest muscles cannot expand enough. This creates problems with air flow, mostly due to lower lung volumes.

PFT can be done with two methods. These two methods may be used together and perform different tests, depending on the information a healthcare provider is looking for. The first method is spirometry—a spirometer is a device with a mouthpiece hooked up to a small electronic machine. The other method is plethysmography, wherein a patient sits or stands inside an air-tight box that looks like a short, square telephone booth to do the tests.

PFT measures: tidal volume (VT)—the amount of air inhaled or exhaled during normal breathing; minute volume (MV)—the total amount of air exhaled per minute; vital capacity (VC)—the total volume of air that can be exhaled after inhaling as much as one can; functional residual capacity (FRC)—the amount of air left in lungs after exhaling normally; residual volume—the amount of air left in the lungs after exhaling as much as one can; total lung capacity—the total volume of the lungs when filled with as much air as possible; forced vital capacity (FVC)—the amount of air exhaled forcefully and quickly after inhaling as much as one can; forced expiratory volume (FEV)—the amount of air expired during the first, second, and third seconds of the FVC test; forced expiratory flow (FEF)—the average rate of flow during the middle half of the FVC test; and peak expiratory flow rate (PEFR)—the fastest rate that one can force air out of one's lungs.

Normal values for PFTs vary from person to person. The amount of air inhaled and exhaled in test results are compared to the average for someone of the same age, height, sex, and race. Results are also compared to any previous test results. If one has abnormal PFT measurements or if the results have changed, one may need other tests.

Forced spirometry consists of having the patient blow through a tube of known dimensions and measuring characteristics of the air forced through the tube such as the velocity and volume of airflow over time. Both numerical and graphic data can be captured and displayed for interpretation of possible lung disease. The forced spirometry test requires a motivated patient and a strong expiratory breathing effort to obtain accurate lung function measurements.

In addition, oscillometry is a relatively new technology to measure pulmonary function that superimposes small air pressure perturbations on the normal quiet breathing of an individual. This approach places less demands on the patient to perform the complete forced expiration required in spirometry. Oscillometry has been particularly helpful in testing pulmonary function in children who may not be motivated to give a strong effort or may not fully understand what is being asked of them for the spirometry test. Oscillometry may also be effective in testing children and adults who are experiencing shortness of breath at the time of testing.

In recent years, portable pulmonary function testing has been made possible with small hand-held devices that can wirelessly transmit digital information to portable computers, smart phones, and other devices for display to allow real-time patient assessment, diagnosis, and medical management.

According to the Centers for Disease Control there are approximately twenty-five (25) million Americans with asthma and fifteen (15) million Americans with COPD. COPD is the third most common cause of death in the U.S. Direct health care expenditures for COPD are approximately thirty (30) billion annually. This includes almost seven (7) million visits to hospital emergency departments alone. Accordingly, it is an object of the present invention to provide a portable device for receiving PFT data, displaying, and analyzing same with both real-time and recorded patient clinical data such as that obtained with ultrasound or an electronic stethoscope. The current disclosure would be an important advance in the diagnosis and medical management of this very large population of patients that consume a great deal of healthcare resources.

### BRIEF SUMMARY OF THE INVENTION

The above objectives are accomplished according to the present invention by providing in a first embodiment a method for gathering and collating diverse medical data. The method includes gathering first medical data with a first device, transmitting the gathered data to a second device, combining the first medical data with at least one second medical data, employing AI to analyze the combined first medical data and the at least one second medical data, displaying the combined first medical data and the at least one second medical data, and creating a personalized diagnosis based on the combined first medical data and the at least one second medical data. Still further, the at least one second medical data is not the same medical data as the gathered data. Further, yet, the at least one second medical data was gathered prior to the first medical data. Yet again, the at least one second medical data was gathered contemporaneously with the first medical data. Furthermore, the method combines the first medical data and the at least one second medical data with genetics information prior to AI analysis. Still again, the combined first medical data and the at least one second medical data may be displayed on a body template view. Still yet, the display may be resolved from the body template view showing a full body template to a single organ view.

In a further embodiment, a portable device for receiving pulmonary function test data as well as displaying, and analyzing the data. The device may include a probe, a transducer, a beamformer, a controller, a signal processor, a B mode processor, a Doppler processor and a video processor. Further, the device may include a transmitter to send recorded pulmonary function test data to another device for analysis. Still, the device may detect various data points, including ultrasound, pulmonary function test results, lung sounds, heart sounds, electrocardiogram (ECG/EKG), blood oxygenation level, blood pressure readings, or body temperature. Again, the device may combine the detected data points with genetics information. Still yet, the device may include a scan converter. Further again, the device may include a body template view to display analytical data. Still yet, the body template view may resolve from a full body template to a single organ view.

In an alternative embodiment, a method for diagnosing dyspnea is provided. The method may include receiving pulmonary function test data, combining the pulmonary function test data with other clinical data, interpreting the combined pulmonary function test data and the other clinical data, and archiving the combined pulmonary function test data and the other clinical data. Still, the method may perform the interpretation using artificial intelligence assistance. Yet again, the method may include recording the interpreted data. Again still, the method may include transmitting the interpreted data to a patient's medical record. Further, the method integrating the combined pulmonary function test data and the other clinical data with previously recorded clinical data. Again further, the other clinical data may include ultrasound, lung sound, heart sound, electrocardiogram (ECG/EKG), blood oxygenation level, blood pressure readings, or body temperature. Yet again, the method may include interpreting the combined pulmonary function test data and the other clinical data in combination with genetics data. Still further, the resultant images derived from at least the combined pulmonary function test data and the other clinical data may be displayed. Further again, the method may include displaying the resultant images on a body template. Yet still, the body template may resolve from a full body template to a single organ view.

In a still further embodiment, a method for analyzing pulmonary function test data is provided. The method may include obtaining digital pulmonary function test data via a probe, obtaining patient physiological data in addition to the pulmonary function test data, recording the obtained pulmonary function test data and patient physiological data, combining the pulmonary function test data and patient physiological data, analyzing the combined pulmonary function test data and patient physiological data, displaying the combined pulmonary function test data and patient physiological data, and creating a personalized diagnosis based on the combined pulmonary function test data and patient physiological data. Still again, the method may include analyzing the combined pulmonary function test data and patient physiological data via artificial intelligence analysis. Yet further, the method may comprise incorporating extraneous data previously obtained with respect to a subject from which the combined pulmonary function test data and patient physiological data was obtained.

### BRIEF DESCRIPTION OF THE DRAWINGS

The construction designed to carry out the invention will hereinafter be described, together with other features thereof. The invention will be more readily understood from a reading of the following specification and by reference to the accompanying drawings forming a part thereof, wherein an example of the invention is shown and wherein:

FIG. 1 shows one embodiment of an ultrasound probe of the current disclosure.

FIG. 2 shows one possible embodiment of a visual display that may display data gathered by an ultrasound probe of the current disclosure.

FIG. 3 illustrates in block diagram form an embodiment of an ultrasonic diagnostic imaging system constructed in accordance with the principles of the current disclosure.

FIG. 4 is a schematic diagram of a diagnosis method of the current disclosure.

FIG. 5 shows one example of a display and body template that may be used as part of the current disclosure.

FIG. 6 shows a view of FIG. 5 with the full body template view resolved to a single organ view of a heart.

It will be understood by those skilled in the art that one or more aspects of this invention can meet certain objectives, while one or more other aspects can meet certain other objectives. Each objective may not apply equally, in all its respects, to every aspect of this invention. As such, the preceding objects can be viewed in the alternative with respect to any one aspect of this invention. These and other objects and features of the invention will become more fully apparent when the following detailed description is read in conjunction with the accompanying figures and examples. However, it is to be understood that both the foregoing summary of the invention and the following detailed description are of a preferred embodiment and not restrictive of the invention or other alternate embodiments of the invention. In particular, while the invention is described herein with reference to a number of specific embodiments, it will be appreciated that the description is illustrative of the invention and is not constructed as limiting of the invention. Various modifications and applications may occur to those who are skilled in the art, without departing from the spirit and the scope of the invention, as described by the appended claims. Likewise, other objects, features, benefits and advantages of the present invention will be apparent from this summary and certain embodiments described below, and will be readily apparent to those skilled in the art. Such objects, features, benefits and advantages will be apparent from the above in conjunction with the accompanying examples, data, figures and all reasonable inferences to be drawn therefrom, alone or with consideration of the references incorporated herein.

### DETAILED DESCRIPTION OF A PREFERRED EMBODIMENT

With reference to the drawings, the invention will now be described in more detail. Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are herein described.

Unless specifically stated, terms and phrases used in this document, and variations thereof, unless otherwise expressly stated, should be construed as open ended as opposed to limiting. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise.

Furthermore, although items, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

The current disclosure combines digital lung information from pulmonary function tests (PFT) with ultrasound and/or other clinical devices. Almost any digital device that provides clinical data like sounds and graphics from an electronic stethoscope, respiratory cycle data from a respiratory detection device, EKG, Pulse Oxygenation, Blood Pressure readings, temperature, etc., —both presently available and those that come on the market in the future. The current disclosure may incorporate combining various clinical data with PFT data to devices other than an ultrasound machine such as an electronic stethoscope or a standalone tablet, smart phone or computer and not something attached to or included in another clinical device, to better diagnosis and manage patients with potential lung problems. Medical data analysis and artificial intelligence software may be used. Artificial Intelligence/Machine Learning medical software could be used to analyze the data and propose a diagnosis. Examples include IBM Watson (https://www.ibm.com/watson/health/), Isabel (https://www.isabelhealthcare.com/), and Human Dx (https://www.humandx.org/). (Google, Microsoft, etc). The combined data will be available for display to the healthcare provider in real-time and/or recorded for later review. It can also be analyzed by artificial intelligence and deep learning to enhance diagnostic accuracy and patient management and produce a personalized patient profile. In addition to patient care, the combination of the data and its analysis can be used to teach health professionals at all levels.

The device of the current disclosure will help address the difficult and important diagnostic question of the cause of a patient's dyspnea or shortness of breath. Combining lung function data from PFTs with other important physiological data such as heart function from heart ultrasound (ECHO), heart and lung sounds from an electronic stethoscope, and EKG in real-time or asynchronously and analyzing with artificial intelligence will be a tremendous advancement in identifying the cause of dyspnea.

This invention will wirelessly, or via a wired connection, combine portable PFT data with both real-time and recorded patient clinical data such as that obtained with ultrasound. It can also be combined with other real-time and recorded patient information such as stethoscope sounds of the heart and lungs, EKG, pulse oxygenation, respiratory cycle, the patient's unique genetic profile, and past medical history. This will allow a more accurate and personalized diagnosis of the patient's dyspnea.

Artificial Intelligence (AI) and deep learning can also be used to further enhance the accuracy of the diagnosis and create a personalized patient profile to better diagnosis and manage health issues in an individual as they arise. AI automates repetitive learning and discovery through data. But AI is different from hardware-driven, robotic automation. Instead of automating manual tasks, AI performs frequent, high-volume, computerized tasks reliably and without fatigue. For this type of automation, human inquiry is still essential to set up the system and ask the right questions, and select relevant data to be included.

AI adds intelligence to existing products. In most cases, AI will not be sold as an individual application. Rather, products are improved with AI capabilities, much like Siri was added as a feature to a new generation of Apple products. Automation, conversational platforms, bots and smart machines can be combined with large amounts of data to improve many technologies at home and in the workplace, from security intelligence to investment analysis.

AI adapts through progressive learning algorithms to let the data do the programming. AI finds structure and regularities in data so that the algorithm acquires a skill. The algorithm becomes a classifier or a predictor. So, just as the algorithm can teach itself how to play chess, in the commercial world it can teach itself what product to recommend next online. And the models adapt when given new data. Back propagation is an AI technique that allows the model to adjust, through training and added data, when the first answer is not quite right.

AI analyzes more and deeper data using neural networks that have many hidden layers. Building a fraud detection system with five hidden layers was almost impossible a few years ago. All that has changed with incredible computer power and big data. You need lots of data to train deep learning models because they learn directly from the data. The more data you can feed them, the more accurate they become.

AI achieves incredible accuracy though deep neural networks—which was previously impossible. For example, interactions with Alexa, Google Search and Google Photos are all based on deep learning—and they keep getting more accurate the more we use them. In the medical field, AI techniques from deep learning, image classification and object recognition can now be used to find cancer on MRIs with the same accuracy as highly trained radiologists.

AI gets the most out of data. When algorithms are self-learning, the data itself can become intellectual property. The answers are in the data. One just has to apply AI to get them out. Since the role of the data is now more important than ever before, it can create a competitive advantage. If one has the best data in a competitive industry, even if everyone is applying similar techniques, the best data will win.

In one embodiment, an ultrasound probe, multi-functional probe, electronic stethoscope, lung assessment smart devices such as spirometers, smart medical devices such as an electronic stethoscope, a digital stethoscope, portable laptop EKGs, or other monitoring/computational device such as a smart phone, tablet, or laptop computer will receive the digital PFT information for display and analysis and combine it with other clinical data for interpretation by the healthcare provider with or without artificial intelligence assistance. This data can also be recorded and transmitted elsewhere for archiving in the patient's medical record or review by a medical consultant. For purposes of example only, smart devices may have display screens for heart sound graphs, lung volume capacity, EKG readings, etc., obtained from a probe and transmitted to the device. E.g., PFT data could be feed into a smart device or data, such as that gathered by an e-stethoscope, could be sent to a tablet or smartphone to combine with the PFT data for analysis and display. The current disclosure has a wide application. For instance, a portable EKG or a pulse oxygenation device which are also wireless and have digital data, as well as display capability, may gather information from a patient and send their respective digital data elsewhere for combination with other patient data and AI analysis.

In one aspect, the current disclosure provides an analytical method approach that is applied to a patient with shortness of breath via combining PFT data with other real-time and recorded patient data and analyzing it with or without artificial intelligence and deep learning. This is unique in medical practice. This will be a significant advancement in diagnosing and treating patients with shortness of breath which is a common and serious clinical scenario globally.

Further, the current disclosure may prove useful in tele-medicine when medical consultation is needed remotely. The combination of PFT data and other important clinical data could be available to the consultant in real-time or asynchronously. In addition to use in clinical medicine, the current disclosure will be an excellent teaching tool for all health professionals. It can be used at the patient's bedside for teaching as well as in medical teaching laboratories and medical simulation and clinical skills centers. The learner can assess and diagnose the patient without AI assistance. Then AI can be activated and results of the learner with and without AI assistance compared. Feedback explanations of any differences can be provided to the learner. Recording of the PFT data and other clinical information will also be useful in developing printed and electronic learning material for learners in the form of case-based instructional problems and other learning approaches.

The current disclosure could be incorporated into almost every healthcare delivery site that cares for patients with obstructive lung disease that present with dyspnea as a chief complaint. This would include thousands of emergency departments, outpatient clinics, hospitals, acute care facilities, and facilities using tele-medicine consultation. In addition, as a teaching tool this invention could be used in training almost all healthcare providers including physicians, nurses, nurse practitioners, physician assistants, emergency medical technicians, and others.

There is presently nothing available that combines pulmonary function test data with a variety of other relevant clinical data to address the common serious symptom of dyspnea. Assessing PFT results and other clinical data either by the healthcare provider alone or with the aid of artificial intelligence and deep learning will provide a distinct advantage whether coupled with an ultrasound machine, an electronic stethoscope or a multi-modality device. This would also provide a distinct advantage for those producers of AI and Deep Learning Software. The same would be true for companies in medical education using advanced technology as they would be able to provide their learners a unique learning experience to enhance their clinical knowledge and their clinical reasoning and diagnostic skills based on AI feedback of common clinical scenarios.

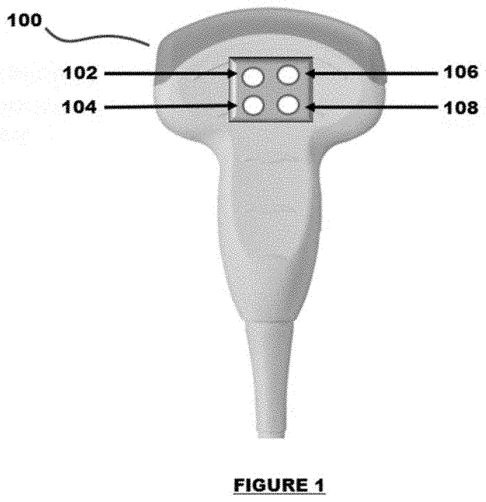

FIG. 1 shows one embodiment of an ultrasound probe 100 capable of receiving PFT Digital Data, Record same, Activate AI, and connect via Wi-Fi, or other means known to those of skill in the art, to other devices such as a computer and/or clinical monitors, not shown. Further examples of probes may include, but are not limited to an electronic stethoscope or other medical diagnostic tool or a non-medical device such as a smart phone or other communications device. The devices that receive the information may also be an electronic stethoscope or other medical diagnostic tool or a non-medical device such as a smart phone or other communications device. Ultrasound probe 100 may include an AI activation switch 102, a Display Monitor activation switch 104, a Wi-Fi activation switch 106, as well as a record activation switch 108. In another embodiment, the PFT data could go to the ultrasound console or smart phone, tablet, or laptop computer for combination with other real-time, synchronized, or recorded patient data and analysis with or without AI assistance.

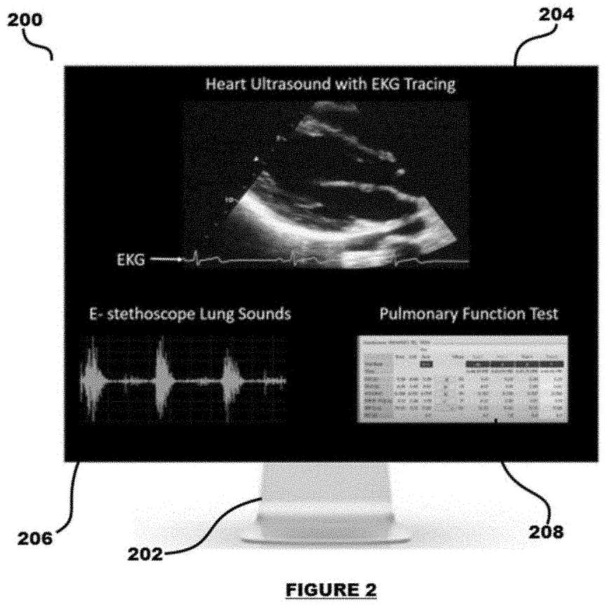

FIG. 2 shows one possible embodiment of a visual display 200 that may display data gathered by ultrasound probe 100 and displayed on a monitor 204 or other display as known to those of skill in the art. As visual display 200 illustrates, multiple iterations of data received from ultrasound probe 100 may be displayed, including ultrasound 204, EKG 206, pulmonary function test results 208, and lung sounds from an electronic stethoscope. The current disclosure is very versatile. Other potential data that may be displayed and combined with the pulmonary function test results for analysis include, but are not limited to, ultrasound images and loops, electronic stethoscope lung sounds, heart sounds, and other sounds, electrocardiogram (ECG/EKG), blood oxygenation level, blood pressure readings, body temperature, body weight, individual and family genetic information, recorded medical history and examination data, e.g., health conditions, allergies, medications, etc., as well as other medical data. The versatility of the current disclosure is especially highlighted by the potential to use it in combination with data such as lung sounds and genetics information.

Differentiating among causes of shortness of breath and wheezing can be difficult, but integrating genetic information into the analysis of PFTs, ultrasound, and EKG data could potentially help discriminate among the varied lung diseases that cause such findings. Information about a particular genomic profile that would increase risk for obstructive lung disease (e.g. asthma or COPD) could aid in interpretation of PFTs by the analytic device. A genetic risk for asthma or COPD could increase the diagnostic accuracy by the device, for example.

Further, little is known about the genetic risk for many common diseases. For example, asthma is thought to result from multiple complex interactions between genetic and environmental influences. Some studies of families of people with asthma (performed before current genomics technology) suggest a heritability of asthma of up to 80 percent, but the varied manifestations of the disease do not follow simple inheritance patterns. Integrating genetic information with the results of the PFTs obtained by the device by researchers could possibly help elucidate different patterns of disease that correlate with modern genomic analyses.

Some diseases, such as cystic fibrosis are monogenic (single gene) disorders that are heritable, but it would be helpful to better correlate pulmonary function testing with existing genomic knowledge of these diseases. This may help tailor diagnostic and treatment strategies for such patients, as different gene mutations that cause the same disease may have different patterns as interpreted by the device.

The device could also be used to help track individuals at increased genetic risk over time to help predict or prognosticate disease. For example, little is known about the relationship about human susceptibility to the development of COPD, but one study suggested that first-degree relatives of individuals with early-onset COPD may have enhanced susceptibility to the harmful effects of cigarette smoking. The device would be able to be used in research studies to more easily track different pulmonary function profiles in people with COPD to genomic profiles, potentially assisting in identifying and tracking individuals at increased risk for developing this disease.

Similarly, there are genetic cardiac disorders that can cause dyspnea such as hypertrophic cardiomyopathy. Genetic testing can help identify patients and family members with this disease. Coronary artery disease is a major cause of heart failure and dyspnea and genetic risk factors have also been identified for coronary artery disease. Thus, in both of these conditions having PFTs with the patient's genetic profile and other physiological data such as that from ultrasound of the heart and an EKG can increase the accuracy of the diagnosis and contribute to the development of a personalized patient treatment plan, especially when the data is analyzed with artificial intelligence. These combinations and analysis of this type of data can also serve as a research method to help us better understand the pathophysiology of these medical conditions.

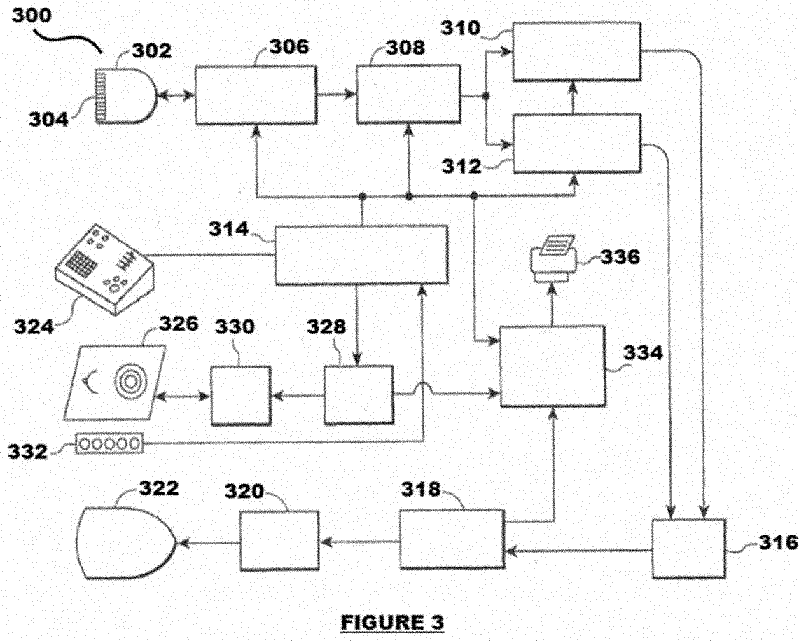

FIG. 3 shows one embodiment via a block diagram of an ultrasound system 300 constructed in accordance with the principles of the current disclosure. A probe 302 has a multi-element array transducer 304 which transmits ultrasound waves into a subject and receives echo signals. The echo signals are converted into electrical signals by the transducer elements and coupled to a beamformer 306. Preferably the transducer signals are digitized and processed digitally in the beamformer. The beamformer forms coherent echo signals when then undergo processing by a signal processor 308 such as quadrature detection, wall filtering (for Doppler signals), or other filtering for signal enhancement such as harmonic signal separation or spatial or frequency compounding. The processed signals are then coupled to a B mode processor 310, such as a B-mode or 2D mode wherein a linear array of transducers simultaneously scans a plane through the body that can be viewed as a two-dimensional image on screen (more commonly known as 2D mode) for B mode imaging which involves the envelope detection of signals from tissue structure, or are processed by a Doppler processor 312, that employs ultrasonography of the Doppler effect to assess whether objects, such as blood, air flow, etc., are moving towards or away from the probe, and the relative velocity of these flows, by calculating the frequency shift of a particular sample volume, for example flow in an artery or a jet of blood flow over a heart valve, its speed and direction can be determined and visualized. For instance, the current disclosure may employ Color Doppler—the measurement of velocity by color scale. Color Doppler images are generally combined with gray scale (B-mode) images to display duplex ultrasonography images to produce images of a patient and the PFT information resulting therefrom. The coordination of the beamformer 306, the signal processor 308, the B mode and Doppler processor 312, as well as the other processing steps in the ultrasound signal path prior to display such as scan conversion, is performed by a controller 314, such as a hardware device or a software program that manages or directs the flow of data between two entities, such as cards, microchips or separate hardware devices for the control of a peripheral device. The resultant 2D or 3D tissue, motion or spectral Doppler image signals are arranged in the desired display format by a scan converter 316. The ultrasound images are then coupled to a memory 318 where an entire sequence of real time images may be captured and replayed for diagnosis. Individual images or a loop (sequence) of images may be stored in an image store (not shown) for later further diagnosis. The images in memory 318 are applied to a video processor 320 which drives the image display 322 in the appropriate manner for display of the images.

The ultrasound system may be operated by a user who manipulates the appropriate controls of a control panel 324 and/or probe 302. Signals from control panel 324 are received by the controller 314 which responds by controlling the ultrasound system as desired by the operator. In accordance with the present invention, the user can use the control panel to call up a body marker template for display on the touchscreen display 326. Multiple inputs may be funneled into display 326 to allow for simultaneous viewing of multiple patient information statistics and readings. Alternately, the ultrasound system may call up a particular template in correspondence with selection by the user of a particular ultrasound exam. For instance, if the user indicates that a heart, or other, exam will be performed, the ultrasound system may call up the templates for a heart, or other, diagnosis. Thus, the body template may further resolve the image from a full body template view to a view narrowed to a single organ or particular locus on the body. The graphical templates are applied to a graphics generator 328, which applies the graphics signals to a touchscreen controller 330, which drives the touchscreen display appropriately to display the selected template. Located below the touchscreen display may be a row of control knobs 332 which are coupled to controller 314 so that signals from control knobs 332 can be received by controller 314. A report generator 334 is also resident on the system 300 and assists in the assembly of a diagnostic report for the examination. Report generator 334 is also controlled by controller 314. Report generator 334 can access ultrasound images from memory 318 and body marker templates from graphics generator 328 for assembly into a diagnostic report. The diagnostic report can be viewed on one of the display screens and/or printed on a printer or other output device, such as a mobile phone, screen, computer, etc., 336.

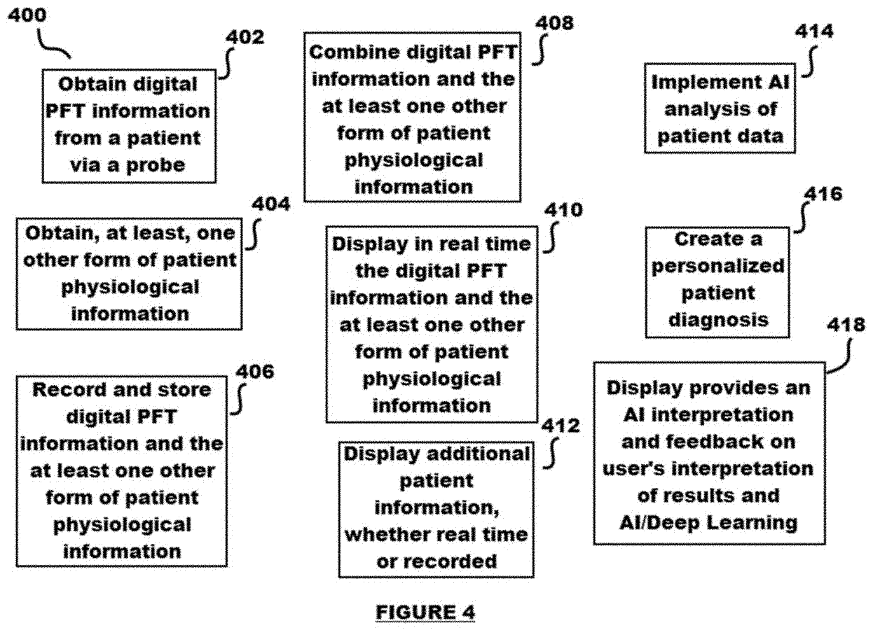

FIG. 4 illustrates a schematic of one embodiment of a method 400 for using a probe of the current disclosure. At step 402, one may obtain digital PFT information from a patient via a probe of the present disclosure. This PFT information may include lung volume, lung capacity, rates of air flow as air is exhaled from the lungs, among other measurements of a patient's pulmonary function. These measurements include tidal volume, minute volume, vital capacity, functional residual capacity, residual volume, total lung capacity, forced vital capacity, forced expiratory volumes, forced expiratory flow, peak expiratory flow rate, response to bronchodilators, diffusion capacity, inspiratory mouth pressures, and expiratory mouth pressures. At step 404, at least one other form of patient physiological information is obtained, this may include, but is not limited to with the following listed for purposes of example only, stethoscope sounds of the heart and lungs, EKG, pulse oxygenation, respiratory cycle. At step 406, this information is recorded and stored. At step 408, the digital PFT information and the at least one other form of patient physiological information are combined. “Combined” meaning the information from various clinical devices/tests/historical information is combined for analysis by the clinician with or without AI assistance. So a particular set of clinical data, for instance, from the ultrasound of the heart in a patient with dyspnea is interpreted then this information in combined with the data from the PFT to give a final more accurate diagnosis of the dyspnea, the severity of the situation, and potential treatment strategies for the patient.

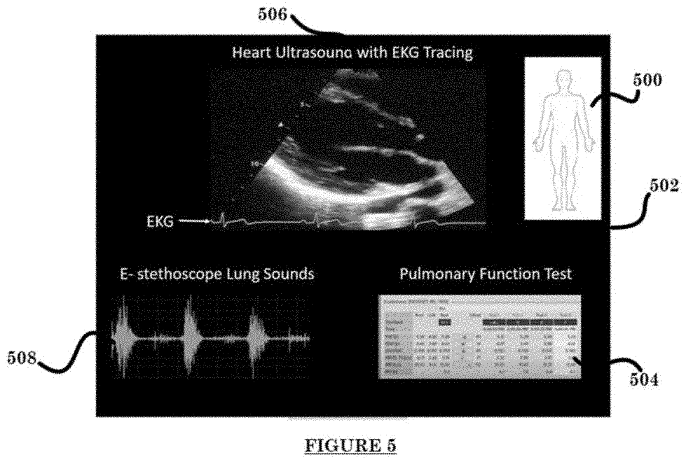

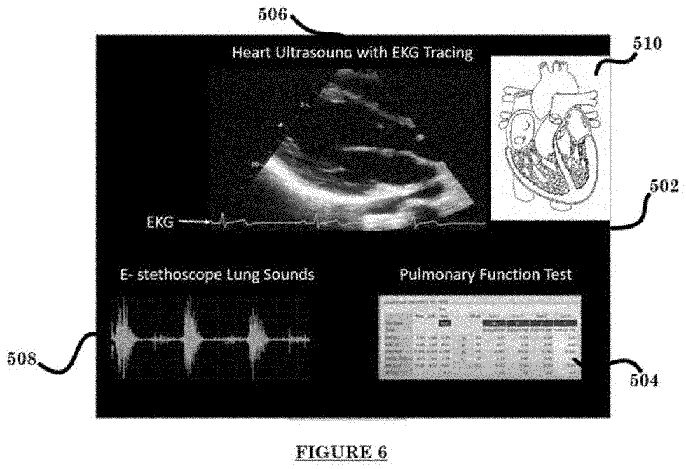

FIG. 5 shows one example of a body template 500 that may be used with a display 502 showing PFT data 504 and input 506 from a medical device, all shown on a display 508, such as an electronic stethoscope providing lung sounds. While template 500 shows a mock-up of a human body, the template may be further resolved to show specific organs and impact of the data on same, e.g., showing a display of lungs that visual air flow and restrictions to same in the displayed lungs or an image of a heart showing ultrasound results from same. FIG. 6 shows a view of FIG. 5 with the full body template 500 resolved to a single organ view of a heart 510. While the resolution of showing same as a heart is provided, other organs, other sections, partial sections, cross sections, organs, etc., of a body, such as head, leg, arm, neck, chest, abdominal area, lungs, kidney, thoracic spine cross section, etc., are all considered possible views for the current disclosure.

For education purposes and self-directed-learning the learner/student would interpret both sets of clinical data (heart ultrasound and PFT) without AI and make a diagnosis and develop a treatment plan. AI would then activate its diagnosis. Differences in the two diagnosis could be explained and feedback from AI given to enhance the learning experience. At step 410, the digital PFT and at least one other form of patient physiological information are displayed. At step 412, additional patient information or extraneous patient or subject information, such as prior recorded physiological clinical tests, may be combined with and/or displayed with the digital PFT and at least one other form of patient physiological clinical information. At step 414, artificial intelligence analysis, which may be performed by IBM Watson (https://www.ibm.com/watson/health), Isabel (htpps://wwww.isabelhealthcare.com/), and Human Dx (https://www.humandx.org/) as well as others may be applied to any or all of the PFT information, at least one other form of patient physiological clinical information such as ultrasound, and/or additional patient information, such as the patient's unique genetic profile. At step 416, a personalized patient diagnosis may be produced. At step 418, a display may provide the user with an AI interpretation and feedback to compare with the user's interpretation and results and AI/Deep Learning.

Diagnostic information such as a diagnosis of COPD and severity of the condition based on present PFT, and if available, comparisons to previous PFTs. With additional data the relative state of the condition would be personalized as well such as whether the condition is getting worse and what the rate of progression of disease is. Other relevant personalized data would be included such as the circumstances and cause of previous COPD episodes, risk factors, potential triggering events in this patient based on history, including seasonal effects, second-hand smoke and chemicals, medication being used and blood level of certain medicine like theophylline which is used to treat COPD, steroids with route of administration and doses. If the patient has more than one disease (other than COPD), the present information can be combined with other clinical data corresponding to co-morbid diseases such as coronary artery disease (CAD) and heart failure. The profile of their CAD would be combined with their COPD profile to improve the diagnostic accuracy of the present episode of dyspnea. This database could also include changes in the status of the CAD, medications, symptoms, weight gain, exercise tolerance, status of smoking, previous results of ultrasound of the heart, and the patient's unique genetic profile. Other co-morbid diseases if present could be included like diabetes and hypertension. All of this would create the patient's unique health profile and serve as a baseline against which diseases, severity, and treatment could be used to provide personalized or precision medicine for this particular patient.

In a further embodiment, a portable device (such as an ultrasound probe, electronic stethoscope, smart phone or tablet) is capable of accepting digital medical information, such as for purposes of example only and not intended to be limiting, PFT data, gathered by a first device or probe and then wirelessly or with wire connection, transmitted to the portable device. After accepting the patient data, it is combined with other medical data such as ultrasound, EKG, heart sounds, blood work, genetics, and medical record data. This data may then me analyzed with AI for more accurate diagnosis of medical conditions, such as for purposes of example only and note intended to be limiting, dyspnea, and displaying or sending the analyzed data to a monitor for display of the data and images. The analyzed data, or simply the gathered data, can be recorded and sent to the medical record or sent for remote consultation. This will allow a personalized approach to patient diagnosis and treatment. All the data and displays can be used for real-time teaching with and without AI or recorded and used for teaching and learning later.

In a further embodiment, a method for gathering and collating diverse medical data is provided. First medical data is collected with a first device, this may be any time of medical data, such as pulmonary, cardiac, metabolic or other medical data. This data may be transmitted from the first device to a second device, such as a smart phone, smart medical device, computer, etc. The first medical data may be combined with at least one second medical data. This combination may simply be displaying the information together on a single display or analyzing the data in a joint review, such as comparing pulmonary data, the first medical data, with second medical data, such as cardiac, metabolic, bone density, ultrasound, bronchoscopy, X-ray, colonoscopy, computed tomography (CT), cystoscopy, Electrocardiogram (EKG), cystourethrogram, biopsy, or other information, to form a more comprehensive view of the patient's health by combining the results of the data and analyzing same as a whole with respect to the patient. Further, while the disclosure states at least one second medical data, multiple types of data may be included, such as cardiac combined with pulmonary combined with dietary, etc., further still the first and second data may be the same medical data, but the second data was taken prior to or contemporaneously with the first medical data. Combination as used herein means that the data may simply be viewed simultaneously on a display or, preferably, that the data may be integrated with one another to provide a fuller health “picture” of the patient, such as combining venal information with cardiac information to determine a heart volume, blood flow rate, capacity etc., while this specific example is provided, multiple combinations and permutations of data are considered within the scope of this disclosure and are hereby so disclosed. Further, AI, as disclosed supra, may be used to analyze the combined first medical data and the at least one second medical data. After being analyzed, the distinct first medical data and second medical data may be displayed. Further, the first and second medical data may displayed before the analysis if the operator so prefers. Additionally, the combined data is used to create a personalized diagnosis of a medical condition or health status based on the combined first medical data and the at least one second medical data. Further, the at least one second medical data may not be the same medical data as the gathered data, for instance, EKG information as the first medical data and blood flow information as the second medical data. Further, the at least one second medical data may have been gathered prior to gathering the first medical data. In another embodiment, the at least one second medical data may be gathered contemporaneously with the first medical data by a second device and the information combined on the second device or sent to a third device, such as a computer, for processing the two types of information. Another novel feature is that genetics information, as described supra herein, may be combined with the first and second medical data for further analysis to provide a more robust understanding of a medical condition. Indeed, the genetics information may be combined with the first and second medical data prior to analysis or combined after an initial AI analysis as part of a second analysis. The original first and second medical data, the combined first and second medical data, or the further processed after combined first and second medical data (such as the first and second data being processed then combined with genetics data with further processing) may be displayed on a body template view. Further, the body template view may be resolved from a full body template to a single organ view.

While the present subject matter has been described in detail with respect to specific exemplary embodiments and methods thereof, it will be appreciated that those skilled in the art, upon attaining an understanding of the foregoing may readily produce alterations to, variations of, and equivalents to such embodiments. Accordingly, the scope of the present disclosure is by way of example rather than by way of limitation, and the subject disclosure does not preclude inclusion of such modifications, variations and/or additions to the present subject matter as would be readily apparent to one of ordinary skill in the art using the teachings disclosed herein.

What is claimed is:

1. A method for gathering and collating diverse medical data comprising;
gathering first medical data with a first device;
transmitting the gathered data from the first device to a second device;
combining the first medical data with at least one second medical data;
employing AI to analyze the combined first medical data and the at least one second medical data;
displaying the combined first medical data and the at least one second medical data; and
creating a personalized diagnosis based on the combined first medical data and the at least one second medical data.

2. The method of claim 1, wherein the at least one second medical data is not the same medical data as the gathered data.

3. The method of claim 1, wherein the at least one second medical data was gathered prior to the first medical data.

4. The method of claim 1, wherein the at least one second medical data was gathered contemporaneously with the first medical data.

5. The method of claim 1, further comprising combining the first medical data and the at least one second medical data with genetics information prior to AI analysis.

6. The method of claim 1, further comprising displaying the combined first medical data and the at least one second medical data on a body template view.

7. The method of claim 6, further comprising resolving the body template view from a full body template to a single organ view.

8. A method for diagnosing dyspnea comprising:
receiving pulmonary function test data from a probe;
transmitting the pulmonary function data obtained by the probe to another device;
combining the pulmonary function test data with other clinical data;
interpreting the combined pulmonary function test data and the other clinical data; and
archiving the combined pulmonary function test data and the other clinical data.

9. The method of claim 8, wherein interpretation is performed using artificial intelligence assistance.

10. The method of claim 8, further comprising recording the interpreted data.

11. The method of claim 8, further comprising transmitting the interpreted data to a patient's medical record.

12. The method of claim 8, further comprising integrating the combined pulmonary function test data and the other clinical data with previously recorded clinical data.

13. The method of claim 8, wherein the other clinical data includes ultrasound, lung sound, heart sound, electrocardiogram (ECG/EKG), blood oxygenation level, blood pressure readings, or body temperature.

14. The method of claim 8, further comprising interpreting the combined pulmonary function test data and the other clinical data in combination with genetics data.

15. The method of claim 8, further comprising displaying resultant images derived from at least the combined pulmonary function test data and the other clinical data.

16. The method of claim 15, further comprising displaying the resultant images on a body template.

17. The method of 16, wherein the body template resolves from a full body template to a single organ view.

18. A method for analyzing pulmonary function test data comprising;
obtaining digital pulmonary function test data;
transmitting the pulmonary function data obtained to another device;
obtaining patient physiological data in addition to the pulmonary function test data;
recording the obtained pulmonary function test data and patient physiological data;
combining the pulmonary function test data and patient physiological data; analyzing the combined pulmonary function test data and patient physiological data;
displaying the combined pulmonary function test data and patient physiological data; and
creating a personalized diagnosis based on the combined pulmonary function test data and patient physiological data.

19. The method of claim 18, further comprising analyzing the combined pulmonary function test data and patient physiological data via artificial intelligence analysis.

20. The method of claim 18, further comprising incorporating extraneous data previously obtained with respect to a subject from which the combined pulmonary function test data and patient physiological data was obtained.
